(19) United States
(12) Patent Application Publication
(10) Pub. No.: US 2017/0354382 A1
(43) Pub. Date: Dec. 14, 2017

(54) USER INTERFACE FOR DISPLAYING PREDICTIVE CARDIO INFORMATION

(71) Applicant: MEDICAL INFORMATICS CORPORATION, Houston, TX (US)

(72) Inventors: EMMA K. FAUSS, HOUSTON, TX (US); ALEXANDER CSICSERY-RONAY, TAOS, NM (US)

(21) Appl. No.: 15/621,903

(22) Filed: Jun. 13, 2017

Related U.S. Application Data
(60) Provisional application No. 62/349,577, filed on Jun. 13, 2016.

(51) Int. Cl. A61B 5/00; G06F 19/00; A61B 5/11; A61B 5/0205

(52) U.S. Cl. CPC A61B 5/7275; A61B 5/0205; G06F 19/3406; A61B 5/1118

(57) ABSTRACT

A user interface allows clinical personnel to view predictive risk indices and related physiological data. When a risk index indicates a dangerous condition, the user interface may generate an alarm for routing to an appropriate caregiver. In addition to displaying patient-specific data, comparisons may be provided between the patient and a patient population for those signals, allowing easy recognition of deviations from the norm for the patient population.

### TECHNICAL FIELD

The present invention relates to the field of medicine, and in particular to a user interface for displaying predictive cardio information.

### BACKGROUND ART

There is high mortality between stage 1 and stage 2 palliative surgery for patients with hypoplastic left heart syndrome (HLHS). HLHS is a rare congenital heart defect in which the left ventricle of the heart is severely underdeveloped (this leads to the need for the surgeries for parallel circulation physiology). There is currently no measure of risk (or method) to detect deterioration of these patients in clinical practice.

Patients with parallel circulation physiology need to be monitored to alert their care provider to the patient's risk of a critical deterioration (i.e., heart attack). Although predictive measures have been devised to predict the risk of critical deterioration, there have been no ways of providing that information to clinical personnel to allow easy use of the predictive information.

### BRIEF DESCRIPTION OF DRAWINGS

The patent or application file contains at least one drawing executed in color. Copies of this patent or patent application publication with color drawing(s) will be provided by the Office upon request and payment of the necessary fee.

The accompanying drawings, which are incorporated in and constitute a part of this specification, illustrate an implementation of apparatus and methods consistent with the present invention and, together with the detailed description, serve to explain advantages and principles consistent with the invention. In the drawings,

FIGS. 1-4 are screenshots illustrating a user interface of a monitor for displaying predictive information according to one or more embodiments.

FIG. 5 is flowchart illustrating a technique for monitoring patients according to one embodiment.

FIG. 6 is a block diagram of a system for collecting physiological data, generating the predictive information, and displaying the predictive information in a user interface as illustrated in FIGS. 1-4, according to one embodiment.

FIG. 7 is a block diagram of a programmable device used in the system of FIG. 6 according to one embodiment.

### DESCRIPTION OF EMBODIMENTS

In the following description, for purposes of explanation, numerous specific details are set forth in order to provide a thorough understanding of the invention. It will be apparent, however, to one skilled in the art that the invention may be practiced without these specific details. In other instances, structure and devices are shown in block diagram form in order to avoid obscuring the invention. References to numbers without subscripts are understood to reference all instance of subscripts corresponding to the referenced number. Moreover, the language used in this disclosure has been principally selected for readability and instructional purposes, and may not have been selected to delineate or circumscribe the inventive subject matter, resort to the claims being necessary to determine such inventive subject matter. Reference in the specification to “one embodiment” or to “an embodiment” means that a particular feature, structure, or characteristic described in connection with the embodiments is included in at least one embodiment of the invention, and multiple references to “one embodiment” or “an embodiment” should not be understood as necessarily all referring to the same embodiment.

The terms “a,” “an,” and “the” are not intended to refer to a singular entity unless explicitly so defined, but include the general class of which a specific example may be used for illustration. The use of the terms “a” or “an” may therefore mean any number that is at least one, including “one,” “one or more,” “at least one,” and “one or more than one.”

The term “or” means any of the alternatives and any combination of the alternatives, including all of the alternatives, unless the alternatives are explicitly indicated as mutually exclusive.

The phrase “at least one of” when combined with a list of items, means a single item from the list or any combination of items in the list. The phrase does not require all of the listed items unless explicitly so defined.

As used herein, the term “a computer system” means a single computer or a plurality of computers working together to perform the function described as being performed on or by a computer system.

As used herein, the term “processing element” means a single hardware processing element or a plurality of hardware processing elements that together may be programmed to perform the indicated actions. The hardware processing elements may be implemented as virtual hardware processing elements of a virtual programmable device hosted on a physical hardware device. Instructions that when executed program the processing element to perform an action may program any or all of the processing elements to perform the indicated action. Where the processing element is one or more multi-core processors, instructions that when executed program the processing element to perform an action may program any or all of the multiple cores to perform the indicated action.

As used herein, the term “medium” means a single physical medium or a plurality of media that together store the information described as being stored on the medium.

As used herein, the term “memory” means a single memory device or a plurality of memory devices that together store the information described as being stored on the medium. The memory may be any type of storage device, including random access memory, read-only memory, optical and electromechanical disk drives, etc.

As used herein, the term “clinician” means a doctor or nurse assigned to a clinical unit and responsible for care of a patient.

Although some of the following description is written in terms that relate to software or firmware, embodiments can implement the features and functionality described herein in software, firmware, or hardware as desired, including any combination of software, firmware, and hardware. References to daemons, drivers, engines, modules, or routines should not be considered as suggesting a limitation of the embodiment to any type of implementation.

Although described below in terms of a monitor for displaying predictive information for parallel circulation cardio events, the invention is not limited to cardio events, but as predictive information is developed for other types of events, embodiments may be adapted to display the predictive information for those other types of events.

The system described herein allows clinicians to monitor and alert a care provider in real-time to the patient's risk of a critical deterioration (i.e. heart attack) in patients with parallel circulation physiology.

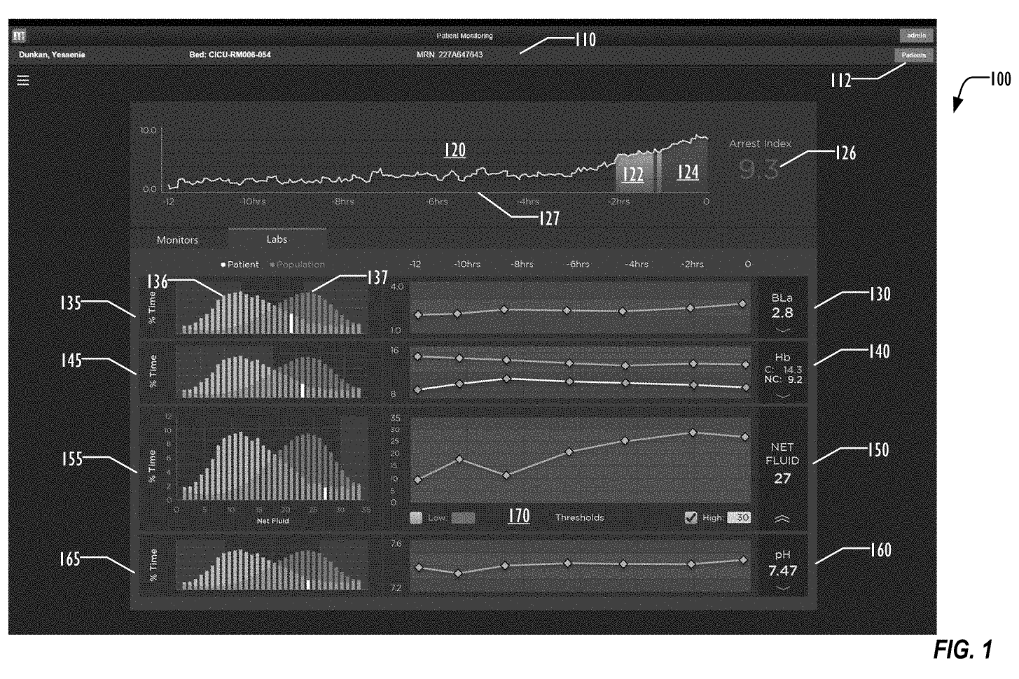

The clinician opens monitor app and selects the appropriate patient. In one embodiment, the app is a part of a clinical information platform system such as the SICKBAY™ Platform provided by Medical Informatics Corp. (SICKBAY is a trademark of Medical Informatics Corp.) From the list of monitor types the clinician selects the cardio monitor. The platform opens a screen like the one illustrated in FIG. 1.

When the monitor is instantiated for the patient, it spins up processes on the platform that does both: (a) a historical calculation for the past 12 hours (or any other desired time period) of the relative risk index (RRI) that is the predictor value; and (b) a real-time calculation for the current RRI value. One technique for calculating the RRI is described in U.S. Pat. Pub. No. 2016/0135756A1, “Clinical Metric for Predicting Onset of Cardiorespiratory Deterioration in Patients,” U.S. patent application Ser. No. 14/942,722, filed Nov. 16, 2015, which is incorporated herein in its entirety for all purposes. The RRI is a type of predictive risk index.

In one embodiment of a graphical user interface (GUI) 100 for the monitoring system illustrated in the screenshot of FIG. 1, a patient bar 110 displays patient identifying information, such as the patient's name, sex, date of birth (DOB), unit and bed number, and patient identification number. A patient select button 112 appears on the right hand of the patient bar 110, allowing the clinician to select the patient to display in the GUI 100. The location and configuration of the patient bar 110 is illustrative and by way of example only, and other locations and configuration of the patient bar 110 may be used as desired.

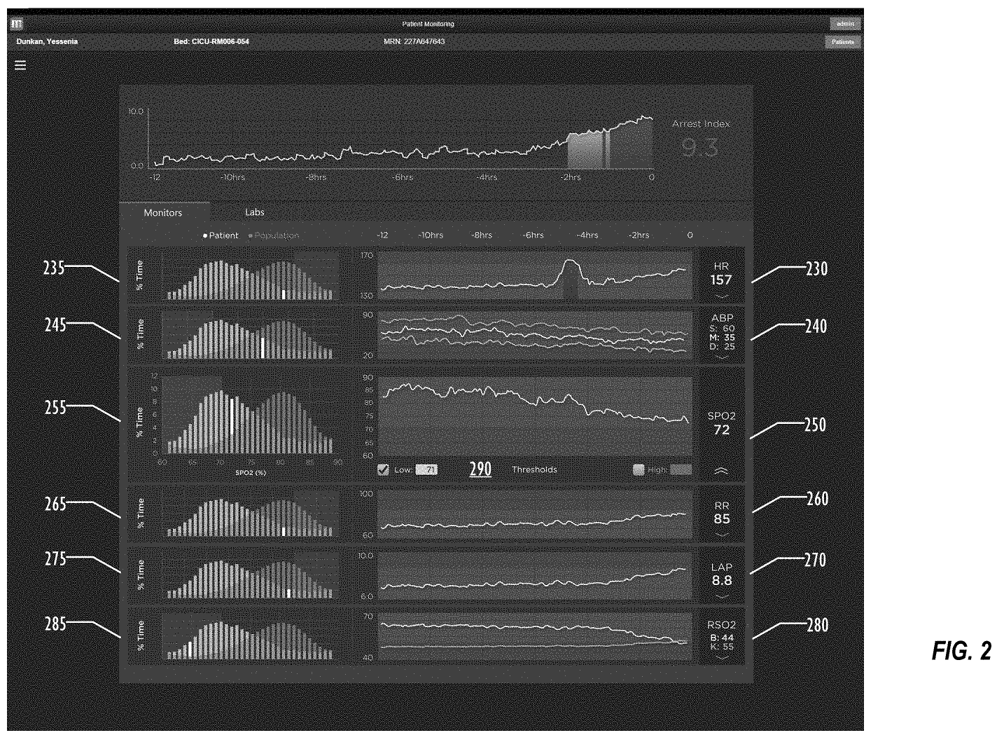

As illustrated in FIGS. 1-2, the GUI 100 also displays a graph 120 that offers real-time monitoring of the patient's condition through use of the RRI and a current value for the RRI 126. The current value and graph are labelled with the name of the arrest index or RRI. The graph display the RRI over a predetermined time duration prior to current time. As illustrated in FIGS. 1-2 the graph of the RRI is a 12 hour record of the RRI. Although only a single RRI is illustrated in FIGS. 1-4, embodiments may define a plurality of RRIs for various conditions and display graphs of that plurality of RRIs in the same GUI 100.

Embodiments may also display a record of presented data signals of interest, preferably with the same time duration as the graph 120. In some embodiments, the signals may include some that are used as algorithm inputs for generating the RRI, however, any other data signals of interest may be displayed. Typically, the data signals shown in this area are data signals that clinical experts have determined are valuable when determining how to respond to the RRI value. In the example of FIG. 1, 12 hour records of lab results are displayed in graphs 130-160, such as beta-lactamase (Bla) (graph 130), hemoglobin (Hb) (graph 140), net fluid (graph 150), and pH (graph 160), based on measurements taken at specific intervals. In the example of FIG. 2, 12 hour records of vital signs monitors are displayed as signal traces, such as heart rate (HR) (graph 230), arterial blood pressure (ABP) (graph 240), oxygen saturation (SPO2) (graph 250), respiratory rate (RR) (graph 260), left atrial pressure (LAP) (graph 270), and regional oxygenation (RSO2) (graph 280). In both FIG. 1 and FIG. 2, the current values of the data are given on the right of the graph. In some embodiments, the GUI 100 may allow the clinician to configure the display to allow the clinician to select desired signals. In other embodiments, the GUI 100 displays automatically selected signals. Although in the examples of FIGS. 1-2 the signals are lab signals or physiological data signals, any type of patient-related signals may be displayed in the GUI 100.

In some embodiments, in which multiple types of RRIs have been defined, the user interface 100 may show multiple RRI graphs. In such an embodiment, a user may be able to click on the RRI graph of interest, which would cause the separate data signals relevant to that RRI to be displayed.

In one embodiment, a hazard indicator may be displayed in the GUI 100, such as on a leftmost portion of the screen, with population percentage indicators appearing alongside the hazard indicator. Displayed on the hazard indicator may an indicator, such as a white box that displays the current RRI value, and is located at the corresponding location on the bar's vertical axis. As the data value changes, the indicator may change in location to match the output.

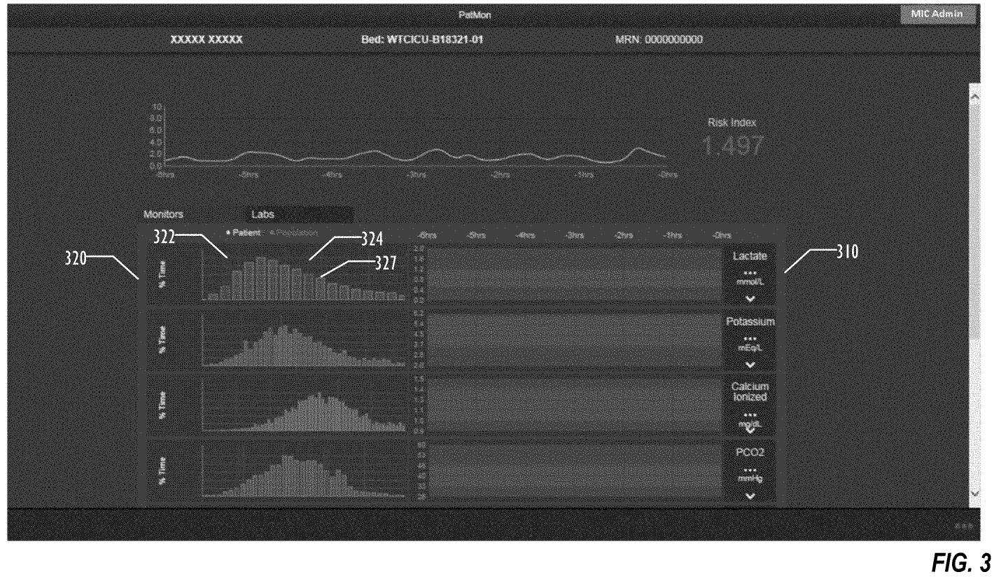

In another embodiment, each of the displayed data signals is accompanied by a histogram showing a distribution of the values of that signal expressed as percentage of time the data had a particular value. In the GUI 100 of FIGS. 1-2, these histograms 135-165 and 235-285 are displayed to the left of the data signals, but other arrangements may be used. In one embodiment, a histogram 137 for an aggregate patient population is joined with the distribution bar chart for the patient 136, allowing the clinician to see easily how the patient's distribution differs from the relevant patient population. Systems may use an aggregate patient population that is specific to the clinical facility, a portion of the clinical facility, or other aggregate populations, such as a national population of patients with similar characteristics. As illustrated in FIG. 3, in some embodiments, where no data for the patient is available in signal are 310, the histogram for the aggregate patient population 327 may be displayed even without a histogram for the specific patient.

In one embodiment, the monitor system GUI 100 may also allow the clinician to set low, high, or both low and high threshold values for a particular physiological signal, which can cause the platform to generate alarms should the patient's data cross the threshold settings. In FIG. 1, the threshold settings are set in a drop-down area, such as drop-down are 170 in which no low threshold is set for net fluid, but a high threshold of 30 is set. Other widgets may be used to allow the clinician to set threshold values as desired. In some embodiments, illustrated in FIGS. 1-4, the threshold values 322 and 324 may be displayed in the histogram area 320 in addition in the signal area, even when the drop down area for setting those threshold values 322-324 is closed and thus invisible in the GUI 100. In FIGS. 1-2, the threshold values are indicated in the histogram area as colored bars marking an area at or above (for high settings) or at or below (for low settings) the threshold value. In FIGS. 1-2, the threshold values are indicated in the histogram area as colored bars or lines at the threshold value. In some embodiments, the threshold values may be illustrated in the signal graph areas by color bars or other graphical indicators.

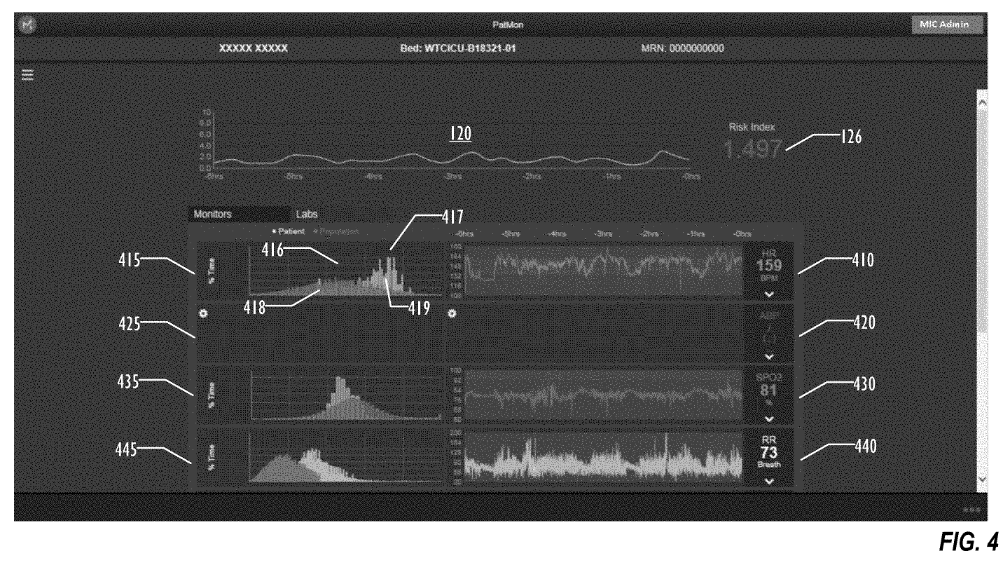

In addition, as illustrated in FIGS. 2 and 4, the GUI 100 may highlight or otherwise indicate on the display when the relevant data signal has crossed one of the thresholds. In FIG. 2, the heartrate (HR) signal exceeded the high threshold for a period between 4 and 6 hours in the past, indicated by a red vertical bar in the HR signal area 230. In FIG. 4, the threshold values in signals 410, 430, and 440 are above the high threshold or below the low threshold a large majority of the time, resulting in a threshold crossing indication over a large portion of the signal areas 410, 430, and 440.

FIG. 4 also indicates that in some situations a desired signal may be unavailable. In this example, signal 420 is related to an Arterial Blood Pressure data signal that is not present, perhaps because the patient managed to dislodge the relevant sensor. In such a situation, an indicator may be placed in the histogram area 425 and signal area 420 instead of the missing signals, making this signal loss condition easy to spot.

In the timeline graph of the RRI illustrated in FIGS. 1-2, a time axis 127 is displayed with 2 hour increments from the current time (t=0) on the right to 12 hours on the left. The increment value is by way of example only, and other increment values may be displayed, such as the 1 hour increments illustrated in FIGS. 3-4. The RRI plot also has a corresponding y-axis that may automatically scale as the metric increases or decreases. In one embodiment, time spent above a critical value may be highlighted to indicate to the user time spent in a more hazardous condition, while time spent below this critical value will not be highlighted.

Risk is defined as a function of hazard and exposure. The RRI provides two views of the data to help the user assess visual representations of both hazard and exposure. In addition to the graph 120, colored areas under the timeline graph 120 may be used to represent the hazard of being at a particular risk value compared to the population. In one embodiment, red indicates that the probability of being at this particular RRI is very low and that the situation is hazardous and yellow indicates a lesser hazard than red, but still one that is of concern. In one embodiment, for example, the red portion of the hazard indication represents less than 5% of the population has experienced this RRI during their treatment. In the example of FIG. 1, the patient began to reach a hazardous RRI value approximately two hours ago, resulting in yellow area 122, and reached a high hazard RRI value approximately one hour ago, resulting in red area 124 that extends to the current time.

The risk to the patient in FIG. 1 generally increases as the RRI index increases over time, first reaching area of concern 122, then reaching the high hazard area 124 more recently. The RRI index is not always monotonically increasing, however, as indicated by the brief bump into red inside the yellow area 122 that returns to the lesser hazard condition after a short time. This visual representation helps the user assess the time a patient has been exposed to deterioration and whether that risk is increasing or decreasing over time.

The RRI score is the relative risk index for a particular patient to have a deterioration event. In one embodiment, a value of 1 is equivalent to the normal risk any patient has of having a deterioration event, a value of 2 represents twice the risk of having a deterioration event, etc. Increasing RRI values represent ever-increasing likelihood of an imminent deterioration event.

If a patient's RRI goes above a certain threshold (for example, the red hazard area illustrated in FIGS. 1-2), in one embodiment an alarm may be generated, preferably with an alarm context message, e.g., “critical deterioration warning in bed 5.” This alarm may be sent off to the clinical alarm system for distribution to assigned caregivers.

By instantiating a real-time monitor where both the real-time and historical risk measure are presented with input signals, that measure may be used as a quality tool to evaluate the effectiveness of interventions such as medications, procedures, and protocols. This can alert a care team to deterioration of HLHS patients, while providing near real-time monitoring for individual patients and optimizing site-specific care by evaluating the effectiveness of various interventions.

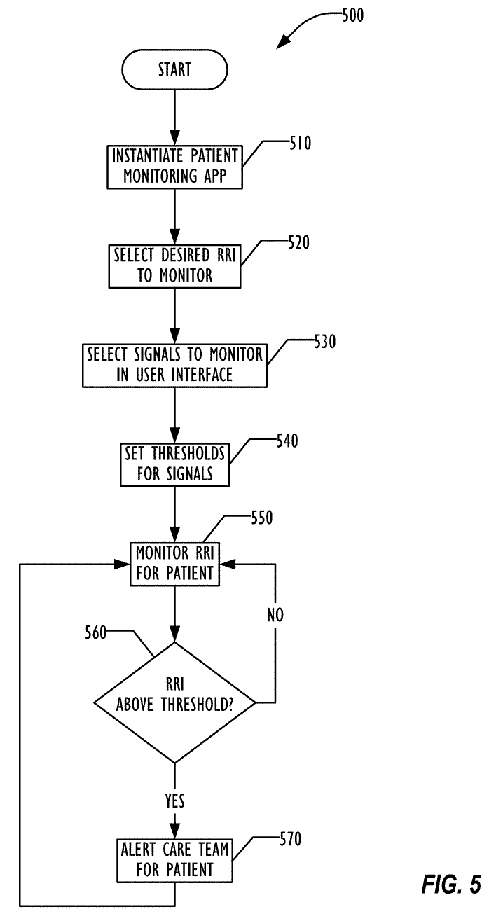

FIG. 5 is a flowchart 500 illustrating a technique for using the monitoring application described above according to one embodiment. In block 510, the monitoring application is instantiated. The monitoring application may be a standalone application or a part of an underlying platform such as the Sickbay platform from Medical Informatics Corp. In block 520, a clinician may select one or more desired RRIs to monitor, if there are more than one RRIs available. If only a single RRI is available, this selection may be omitted. In block 530, the clinician may select signals to monitor in the GUI 100 for the monitoring application. In some embodiments, a default set of signals may be automatically selected and may then be modified by the clinician.

In block 540, the clinician may set thresholds for the signals, as described above. Then in block 550 the monitoring application may display the RRI graphs and signals described above in the GUI 100. In block 560, if the RRI exceeds a threshold for the RRI that indicates a hazardous condition, in block 570 the monitoring application may alarm the patient and send an alarm message to a care team for the patient indicating the presence of the hazardous condition based on the RRI before continuing to monitor the patient.

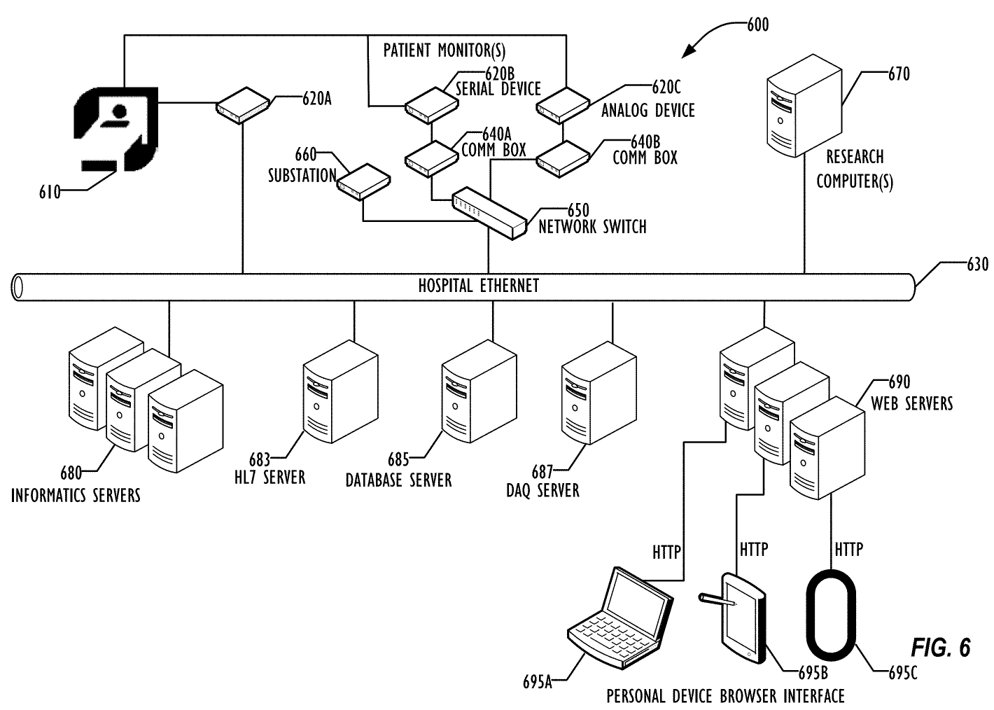

FIG. 6 is a block diagram illustrating a system 600 for collecting, archiving, and processing arbitrary data in a healthcare environment that can deploy a user interface as described above, according to one embodiment.

As illustrated, there are five types of servers: the data acquisition (DAQ) server 687, the informatics server(s) 680, the database server 685, the Health Level 7 (HL7) server 683, and the web server(s) 690. Any number of any of the types of servers may be deployed as desired. All of the servers 680-690 connect to each other and the bedside monitors via one or more hospital networks 630. Although illustrated as a single hospital Ethernet network 630, any number of interconnected networks may be used, using any desired networking protocols and techniques.

Also connected to the hospital network 630 are a number of bedside monitors for monitoring physiological data for a patient in bed 610. These bedside monitors may include network connected monitors 620A, which can deliver digital physiological data to the hospital network 630, serial devices 620B, which produce digital data but are not directly connected to a network, and analog devices 620C, which produce analog data and are not directly connected to a network. Communication boxes 640A and 640B allow connecting the serial devices 620B and analog devices 620C, respectively, to the hospital network 630, typically through a network switch 650. In addition, a substation 660 may be also connected to the network 630 via the network switch 650 for performing data manipulation and time synchronization as described below. Any number of bedside monitor devices 620 may be used as determined advisable by physicians and other clinical staff for the patient in bed 610.

Although as illustrated in FIG. 6 the bedside monitors and associated communication devices are connected directly or indirectly to the hospital network 630, remote bedside monitoring devices may be used as part of the system 600, such as home monitoring devices, connected to the hospital network 630 indirectly through the Internet or through other communication techniques.

Additionally, one or more research computers 670 may be connected, directly or indirectly, to the hospital network 630, allowing researchers to access aggregated data collected from bedside monitors 620 for performing analytics and development.

The web servers 690 are configured for communicating with personal devices such as laptop 695A, tablet 695B, or smart phone 695C via a web browser interface using HyperText Transport Protocol (HTTP).

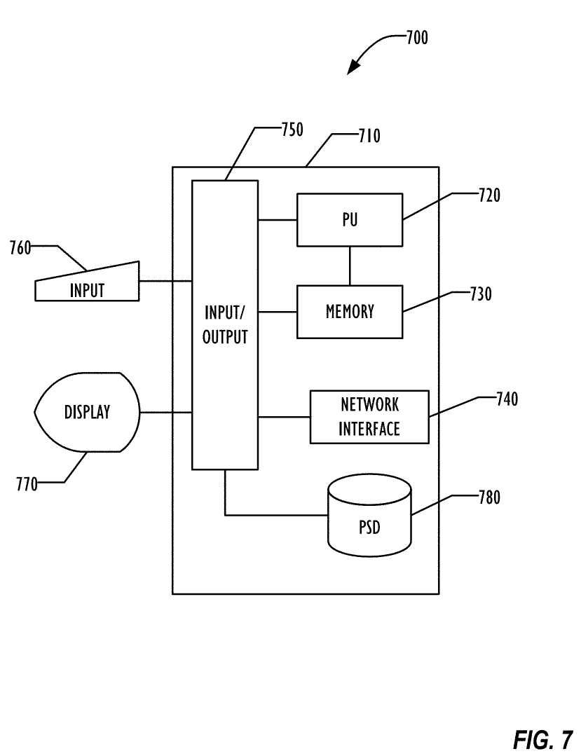

Referring now to FIG. 7, an example computer 700 for use as one of the servers 380-390 is illustrated in block diagram form. Example computer 700 comprises a system unit 710 which may be optionally connected to an input device or system 760 (e.g., keyboard, mouse, touch screen, etc.) and display 770. A program storage device (PSD) 780 (sometimes referred to as a hard disc) is included with the system unit 710. Also included with system unit 710 is a network interface 740 for communication via a network with other computing and corporate infrastructure devices (not shown). Network interface 740 may be included within system unit 710 or be external to system unit 710. In either case, system unit 710 will be communicatively coupled to network interface 740. Program storage device 780 represents any form of non-volatile storage including, but not limited to, all forms of optical and magnetic, including solid-state, storage elements, including removable media, and may be included within system unit 710 or be external to system unit 710. Program storage device 780 may be used for storage of software to control system unit 710, data for use by the computer 700, or both.

System unit 710 may be programmed to perform methods in accordance with this disclosure. System unit 710 comprises a processor unit (PU) 720, input-output (I/O) interface 750 and memory 730. Processor unit 720 may include any programmable controller device, such as microprocessors available from Intel Corp. and other manufacturers. Memory 730 may include one or more memory modules and comprise random access memory (RAM), read only memory (ROM), programmable read only memory (PROM), programmable read-write memory, and solid-state memory. One of ordinary skill in the art will also recognize that PU 720 may also include some internal memory including, for example, cache memory.

Embodiments may be implemented in one or a combination of hardware, firmware, and software. Embodiments may also be implemented as instructions stored on a computer-readable storage medium, which may be read and executed by at least one processing element to perform the operations described herein. A computer-readable storage medium may include any non-transitory mechanism for storing information in a form readable by a machine (e.g., a computer). For example, a computer-readable storage device may include read-only memory (ROM), random-access memory (RAM), magnetic disk storage media, optical storage media, flash-memory devices, and other storage devices and media.

Embodiments, as described herein, may include, or may operate on, logic or a number of components, modules, or mechanisms. Modules may be hardware, software, or firmware communicatively coupled to one or more processing elements in order to carry out the operations described herein. Modules may be hardware modules, and as such, modules may be considered tangible entities capable of performing specified operations and may be configured or arranged in a certain manner. Circuits may be arranged (e.g., internally or with respect to external entities such as other circuits) in a specified manner as a module. The whole or part of one or more programmable devices (e.g., a standalone client or server computer system) or one or more hardware processing elements may be configured by firmware or software (e.g., instructions, an application portion, or an application) as a module that operates to perform specified operations. The software may reside on a computer readable medium. The software, when executed by the underlying hardware of the module, causes the hardware to perform the specified operations. Accordingly, the term hardware module is understood to encompass a tangible entity, be that an entity that is physically constructed, specifically configured (e.g., hardwired), or temporarily (e.g., transitorily) configured (e.g., programmed) to operate in a specified manner or to perform part or all of any operation described herein. Where modules are temporarily configured, each of the modules need not be instantiated at any one moment in time. For example, where the modules comprise a general-purpose hardware processing element configured using software; the general-purpose hardware processing element may be configured as respective different modules at different times. Software may accordingly program a hardware processor, for example, to constitute a particular module at one instance of time and to constitute a different module at a different instance of time. Modules may also be software or firmware modules, which operate to perform the methodologies described herein.

While certain exemplary embodiments have been described in details and shown in the accompanying drawings, it is to be understood that such embodiments are merely illustrative of and not devised without departing from the basic scope thereof, which is determined by the claims that follow.

we claim:

1. A medical patient monitoring system using a predictive risk index, comprising:
one or more processors; and
a memory, coupled to the one or more processors, on which are stored instructions for receiving and displaying historical patient data for a patient and calculating a predictive risk index, comprising instructions that when executed cause the one or more processors to:
receive the historical patient data;
calculate the predictive risk index based on the historical patient data; and
display the predictive risk index and selected historical patient data in a graphical user interface, comprising:
a patient bar, adapted to display patient identifying information;
a predictive risk index area, comprising a graph of the predictive risk index over a predetermined time duration prior to current time and a current value of the predictive risk index; and
a graph of a data signal of the historical patient data associated with the predictive risk index over the predetermined time duration prior to current time.

2. The medical patient monitoring system of claim 1, wherein the graphical user interface further comprises:
a histogram area, adapted to display a first histogram of the data signal over the predetermined time duration prior to the current time.

3. The medical patient monitoring system of claim 2, wherein the histogram area is further adapted to display a second histogram of the data signal for an aggregate patient population.

4. The medical patient monitoring system of claim 1, wherein the instructions further comprise instructions that when executed cause the one or more processors to:
allow setting of a threshold value for the data signal; and
indicate the threshold value for the data signal in the graphical user interface.

5. The medical patient monitoring system of claim 1, wherein the instructions further comprise instructions that when executed cause the one or more processors to display a plurality of predictive risk indices for the patient.

6. The medical patient monitoring system of claim 1, wherein the graph of the predictive risk index comprises an indication that the predictive risk index indicates a hazardous condition exists for the patient.

7. The medical patient monitoring system of claim 1, where the graph of the data signal comprises an indication that the data signal is outside of a threshold value defined for the data signal.

8. A method of monitoring a medical patient based on a predictive risk index, comprising:
selecting a patient to monitor;
receiving historical patient data for the patient;
calculating the predictive risk index for the patient;
displaying a graph of the predictive risk index over a predetermined time duration prior to current time and a current value of the predictive risk index; and
displaying a graph of a data signal associated with the predictive risk index over the predetermined time duration prior to current time.

9. The method of claim 8, further comprising:
displaying a first histogram of the data signal over the predetermined time duration prior to the current time; and
displaying a second histogram of the data signal for an aggregated patient population.

10. The method of claim 8, further comprising:
receiving a threshold value setting for the data signal; and
indicating the threshold value for the data signal.

11. The method of claim 8, further comprising:
displaying a plurality of predictive risk indices for the patient.

12. The method of claim 8, further comprising:
indicating a hazardous condition exists for the patient responsive to the predictive risk index having a value over a predetermined threshold.

13. The method of claim 12, further comprising:
generating an alarm responsive to indicating the existence of the hazardous condition.

14. A non-transitory machine readable medium on which are stored instructions for presenting monitoring information for a medical patient in a graphical user interface, comprising instructions that when executed cause a machine to:
calculate a predictive risk index associated with the medical patient;
display a graph of the predictive risk index over a predetermined time duration prior to current time;
display a current value of the predictive risk index; and
display a graph of a data signal associated with the predictive risk index over the predetermined time duration prior to current time.

15. The machine readable medium of claim 14, wherein the instructions further comprise instructions that when executed cause the machine to:
display patient identifying information in the graphical user interface.

16. The machine readable medium of claim 14, wherein the instructions further comprise instructions that when executed cause the machine to:
display a histogram area in the graphical user interface, in which a first histogram of the data signal over the predetermined time duration prior to the current time is displayed.

17. The machine readable medium of claim 16, wherein the instructions further comprise instructions that when executed cause the machine to:
display a second histogram of the data signal for an aggregate patient population in the histogram area.

18. The machine readable medium of claim 14, wherein the instructions further comprise instructions that when executed cause the machine to:
set a threshold value for the data signal responsive to user input; and
display an indication of the threshold value in the graphical user interface.

19. The machine readable medium of claim 18, wherein the instructions further comprise instructions that when executed cause the machine to:
display an indication that the data signal is outside of the threshold value.

20. The machine readable medium of claim 14, wherein the instructions further comprise instructions that when executed cause the machine to:
display an indication associated with the graph of the predictive risk index that the predictive risk index indicates a hazardous condition exists for the patient.
